(19) United States
(12) Patent Application Publication
(10) Pub. No.: US 2020/0323811 A1
(43) Pub. Date: Oct. 15, 2020

(54) INTRANASAL PHARMACEUTICAL COMPOSITION COMPRISING ANTICANCER DRUGCONTAINING NANOPARTICLES FOR TREATING BRAIN DISEASES

(71) Applicant: INDUSTRY-UNIVERSITY COOPERATION FOUNDATION HANYANG UNIVERSITY, Seoul (KR)

(72) Inventors: Sang-Kyung LEE, Seoul (KR); Kuen Yong LEE, Seoul (KR); Chae Ok YUN, Seoul (KR); Min Hyung LEE, Gyeonggi-do (KR); Kun Ho CHUNG, Seoul (KR); Irfan ULLAH, Seoul (KR)

(21) Appl. No.: 16/304,659

(22) PCT Filed: May 2, 2017
(86) PCT No.: PCT/KR2017/004655

§ 371 Date: Nov. 26, 2018

(30) Foreign Application Priority Data
May 24, 2016 (KR) 10-2016-0063479

(51) Int. Cl. A61K 31/337; A61K 9/16; A61P 35/00; A61K 9/00

(52) U.S. Cl. CPC A61K 31/337; A61K 9/1647; A61K 9/0043; A61K 9/1635; A61P 35/00; A61K 9/1641

(57) ABSTRACT

The present invention relates to an intranasal pharmaceutical composition comprising anticancer drug-containing nanoparticles for treating brain diseases. More specifically, the anticancer drug-containing nanoparticles are nasally administered to deliver the anticancer drug to only brain cells, thereby increasing therapeutic effects on brain tumor and reducing cytotoxicity to normal cells by an organic solvent used in the conventional delivery of the anticancer drug.

### BACKGROUND

### 1. Field of the Invention

The present invention relates to an intranasal pharmaceutical composition comprising anticancer agents-containing nanoparticles for treating brain diseases.

### 2. Discussion of Related Art

Paclitaxel (Taxol), which has been approved by the FDA, is used as an anticancer agent for ovarian cancer, breast cancer, or lung cancer. However, since Taxol binds to the mitotic spindle of segmenting cells to inhibit segmentation of cells, when injected by systemic injection, Taxol reached organ/cells, other than cancer cells, thereby causing many side effects such as hair loss, muscle pain, diarrhea, etc. In addition, since most anticancer agents are hydrophobic, they are administered by systemic injection after being dissolved in an organic solvent, Cremophor EL. While there are serious side effects due to cytotoxicity of the injection, due to a high therapeutic effect of inhibiting replication of cancer cells, despite the side effects, they can be used in clinical practice.

Meanwhile, temozolomide, which is widely used for a brain tumor, is an oral preparation that induces the death of actively differentiating cells, as an alkylating agent binding to cellular DNA. However, because of the blood brain barrier (BBB), temozolomide is insufficiently delivered to a brain tumor, and binds to normal cells, thereby causing many side effects.

Although most known anticancer agents inhibit brain tumor cell division, thereby inhibiting the growth of the brain tumor, they are also disadvantageous in that they cause side effects which affect rapidly dividing normal cells when developed as an oral or injectable preparation.

### SUMMARY OF THE INVENTION

The present invention is directed to providing an intranasal pharmaceutical composition for treating a brain disease by delivering nanoparticles in which an anticancer agent for inhibiting the formation or breakdown of a mitotic spindle is loaded in a nose-to-brain route.

The present invention is also directed to providing a kit for intranasal administration to treat a brain disease, which includes the intranasal pharmaceutical composition for treating a brain disease.

The present invention is also directed to providing a method of treating a brain disease using the intranasal pharmaceutical composition for treating a brain disease.

To achieve the above-described objects, the present invention provides an intranasal pharmaceutical composition, which includes nanoparticles containing an anticancer agent for inhibiting the formation or breakdown of a mitotic spindle.

The present invention also provides a kit for intranasal administration to treat a brain disease, which includes the intranasal pharmaceutical composition for treating a brain disease and a nasal-brain drug delivery system.

The present invention also provides a method of treating a brain disease, which includes intranasally administering the intranasal pharmaceutical composition for treating a brain disease into a subject in need thereof.

In the present invention, nanoparticles containing an anticancer agent for treating a brain disease are intranasally administered to reduce cytotoxicity of normal cells by on organic solvent used in conventional delivery of an anticancer agent and deliver the anticancer agent only to brain cells, thereby increase a therapeutic effect on a brain tumor.

### BRIEF DESCRIPTION OF THE DRAWINGS

FIG. 1 is a schematic diagram illustrating a process of producing paclitaxel (PTX)-loaded nanoparticles (NP-PTX) of the present invention.

FIG. 2A shows an average particle diameter (ZAve) and particle size distribution of NP-PTX, in nanometer scale measured by dynamic light scattering. The polydispersity indexes (PDI) for all tested samples are in an acceptable range (>0.1) (FIG. 2A, inner).

FIG. 2B shows scanning electron microscope (SEM) images of NPs only and NP-PTX (Scale bar: 400 μm

FIG. 2C shows PTX release kinetics from NPs in PBS at 37° C. at each time point. The data is expressed as the mean±SD obtained from experiments performed in triplicate.

FIG. 3A shows an analysis result for viability and apoptosis of C-6 glioma cells according to treatment with various concentrations of PTX only, NP-PTX and RGD-NP-PTX.

FIG. 3B shows ratios of viable and dead C-6 glioma cells according to treatment with various concentrations of PTX only, NP-PTX and RGD-NP-PTX.

FIGS. 4A to 4D show results of inducing an anticancer effect of PTX-loaded NPs in vitro: FIG. 4A shows the anti-proliferation effect measured by CCK-8 analysis after treatment with C6(a) (left) and U87MG(a) (right) glioma cells are treated at the same PTX concentration for 24 hours. The data is expressed as the mean±SD of three independent experiments. FIG. 4B shows the result of flow cytometry of apoptosis in glioma cells after treatment with PTX or NP-PTX for 24 hours. Representative dot plots (upper panel) and accumulated data (lower panel) show the percentages of annexin V and 7AAD. The data is expressed as the mean±SD of three independent experiments. FIG. 4C shows the result of TUNEL analysis. Representative fluorescence microscope images show TUNEL-positive cells (red) and Hoechst-stained nucleus (blue) of glioma cells treated with the same amount of PTX (upper panel). The percentage of TUNEL-positive cells was calculated from 4 or more images per sample using ImageJ software (lower). The data is expressed as the mean±SD of three independent experiments. FIG. 4D shows a result of DNA content analysis when C6 glioblastoma cells are treated with PTX only, NP-PTX or RGD-NP-PTX (representing the percentage of cells inhibited at the G1, S and G2-M phases). Recovered cells are stained with PI and analyzed using a flow cytometer, and data is expressed as the mean±SD obtained from three independent experiments.

FIGS. 5A and 5B show the delivery of NPs to the brain by intranasal inoculation: FIG. 5A shows a bio-distribution of I.N. inoculated alexa488 (A488)-labeled NPs (n=6 per group) inoculated in a rat glioblastoma model. The brain (dorsal view and coronal section view) is examined for the presence of A488 at 24 hours after the inoculation of NP, A488 only, A488-labeled NPs (NP-A488) and A488-labeled RGD-modified NPs (RGD-NP-A488). Representative images representing a relative fluorescent intensity of each indicated organ (right) measured at a certain pixel value for each isolated organ from displayed test cohort±SE (brain dorsal and coronal section views (a, right) and accumulated data (a, left)) are shown. FIG. 5B is a fluorescent microscope image of brain cryosections. The representative images show A488 (green) and Hoechst-stained nuclei (blue) (b; upper panel) and a non-glioblastoma region (b; lower panel) in glioblastoma from the coronal sections shown in a).

FIG. 6 shows a result of confirming the delivery of NPs to an organ.

FIGS. 7A to 7E show the effect of inhibiting in vivo tumor growth by intranasal inoculation of NP-PTX in a mouse glioblastoma model: FIG. 7A shows representative bioluminescence imaging (a, upper panel), an excised brain image (a), middle panel) and ex vivo bioluminescence imaging of a normal or glioblastoma model treated with 2 mg/kg of PBS (Mock) plain NPs and an equivalent dose of PTX only (PTX), PTX-loaded NPs (NP-PTX) and PTX-loaded RGD-modified NPs (RGD-NP-PTX). FIG. 7B shows the bioluminescence intensities (BLI) in vivo (b, left) and ex vivo (b, right) in tested groups shown in FIG. 7A. FIG. 7C shows representative Nissl staining of successive brain sections (upper panel) from and cancer volume in mm³ (lower panel) in the tested groups shown in FIG. 7A. The scale bar represents 100 μm. FIG. 7D shows a representative hematoxylin and eosin-staining result for a paraffin-embedded section from the tested groups shown in FIG. 7A. The scale bar represents 100 μm. FIG. 7E shows a representative image of paraffin-embedded brain section showing TUNEL-positive cells (red) and DAPI-stained nuclei (blue) Ki67 immunostaining (e), lower panel) from the tested group shown in FIG. 7A (e, upper). The scale bar represents 100 μm. Data is expressed as mean±SD (* P<0.05, ** P<0.01, *** P<0.001 and n.s not significant). The data was statistically analyzed using the Mann-Whitney Test for assessing differences in averages between two groups, and by one-way ANOVA for assessing differences in averages between two or more groups using Graphpad Prism 5 software. P<0.05 was considered statistically significant.

FIG. 8 shows a body weight of an animal according to the day after tumor implantation.

FIGS. 9A and 9B show an effect of reducing in vivo tumor growth by intranasal inoculation of NP-PTX in a mouse glioblastoma model: FIG. 9A shows representative live bioluminescence imaging (a, upper) of normal and viable bioluminescence intensities (BLI) in vivo (a, lower) in a normal or glioblastoma model treated with 1 mg/kg PBS (Mock) plain NPs and an equivalent dose of PTX only (PTX), PTX-loaded NPs (NP-PTX) and PTX-loaded RGD-modified NPs (RGD-NP-PTX). FIG. 9B shows a cancer volume (lower panel) in mm³ in the tested groups shown in FIG. 9A.

FIG. 10 is a block diagram explaining a function of a drug delivery system for nose-to-brain administration of the present invention.

FIG. 11 is a diagram illustrating a method of using a drug delivery system for nose-to-brain administration of the present invention.

### DETAILED DESCRIPTION OF EXEMPLARY EMBODIMENTS

Hereinafter, the constitution of the present invention will be described in detail.

The present invention relates to an intranasal pharmaceutical composition for treating a brain disease, wherein the composition includes NPs containing an anticancer agent for inhibiting the formation or breakdown of a mitotic spindle.

In the present invention, since an anticancer agent for inhibiting the formation or breakdown of a mitotic spindle is encapsulated in polymer NPs and administered in a nose-to-brain administration route using a drug delivery system for nose-to-brain delivery, resulting in direct delivery to brain cells, 1) effective segmentation of brain tumor cells is inhibited, 2) there is almost no toxicity of the anticancer agent to normal brain cells because most brain cells are not divided in the G0 phase and the anticancer agent only binds to the divided brain tumor cells to inhibit cell replication, 3) there is no toxicity due to Cremophor EL, which is a conventionally-used organic solvent because the anticancer agent is encapsulated in polymer NPs, and 4) there is a decrease in side effects of the anticancer agent on normal cells except for brain cells by delivering the anticancer agent only to the brain cells through nose-to-brain delivery such that the delivery of the anticancer agent to other organs/cells is minimized, thus a brain tumor therapeutic effect may be increased.

Examples of the anticancer agent for inhibiting the formation or breakdown of a mitotic spindle may include vinca alkaloid-based anticancer agents including vinblastine, vincristine, vinflunine, vindesine and vinorelbine, which inhibit assembly (formation) of the mitotic spindle. In addition, as an anticancer agent for inhibiting the breakdown of the mitotic spindle, a taxane-based anticancer agent such as cabazitaxel, docetaxel, larotaxel, ortataxel, paclitaxel or tesetaxel; or an epothilone-based anticancer agent such as ixabepilone may be used.

The NPs may be prepared from a biodegradable polymer. The biodegradable polymer may be selected from the group consisting of, for example, poly-D-lactic acid, poly-L-lactic acid, poly-D,L-lactic acid, poly-D-lactic acid-co-glycolic acid, poly-L-lactic acid-co-glycolic acid, poly-D,L-lactic acid-co-glycolic acid (PLGA), polylactide (PLA), polylactide-glycolide (PLA/GA), polyalkylcyanoacrylate, poly(acryloyl hydroxyethyl) starch, a copolymer of polybutylene terephthalate-polyethylene glycol, chitosan and a derivative thereof, a copolymer of polyorthoester-polyethylene glycol, a copolymer of polyethyleneglycol terephthalate-polybutylene terephthalate, polysebacic anhydride, pullulan and a derivative thereof, starch and a derivative thereof, cellulose acetate and a derivative thereof, a polyanhydride, polycaprolactone, polycarbonate, polybutadiene, polyester, polyhydroxybutyric acid, polymethyl methacrylate, polymethacrylic acid ester, polyorthoester, polyvinylacetate, polyvinyl alcohol, polyvinyl butyral, polyvinyl formal, albumin, casein, collagen, fibrin, fibrinogen, gelatin, hemoglobin, transferrin, zein and a mixture thereof.

A component targeting a tumor marker may be additionally conjugated to the NPs.

The component targeting the tumor marker may be an RGD peptide or cilengitide, which targets integrin; or an EGF- or EGFR-binding peptide, which is a ligand binding to EGFR.

The anticancer agent-containing NPs may be prepared using a known preparation method. According to an exemplary embodiment of the present invention, the NPs may be prepared by a water-in-oil-in-water (w/o/w) double-emulsion method. Specifically, PLGA and PTX as an anticancer agent are dissolved in an organic solvent, the resulting solution is emulsified through sonication, the single emulsion is re-emulsified in an aqueous PVA solution and sonicated, thereby obtaining a double emulsion, and the double emulsion is added to a PVA solution to evaporate the organic solvent, thereby obtaining the anticancer agent-containing NPs.

The organic solvent may be, for example, dichloromethane, acetone, methylene chloride, ethyl acetate, hexane, and/or tetrahydrofuran.

According to an exemplary embodiment of the present invention, the anticancer agent-containing NPs may have a spherical shape with an average diameter of approximately 150 to 200 nm.

The present invention also relates to a kit for intranasal administration to treat a brain disease, which includes an intranasal pharmaceutical composition for treating a brain disease; and a nose-to-brain drug delivery system.

The intranasal pharmaceutical composition for treating a brain disease according to the present invention may be sprayed in a nose-to-brain route using a drug delivery system for nose-to-brain delivery.

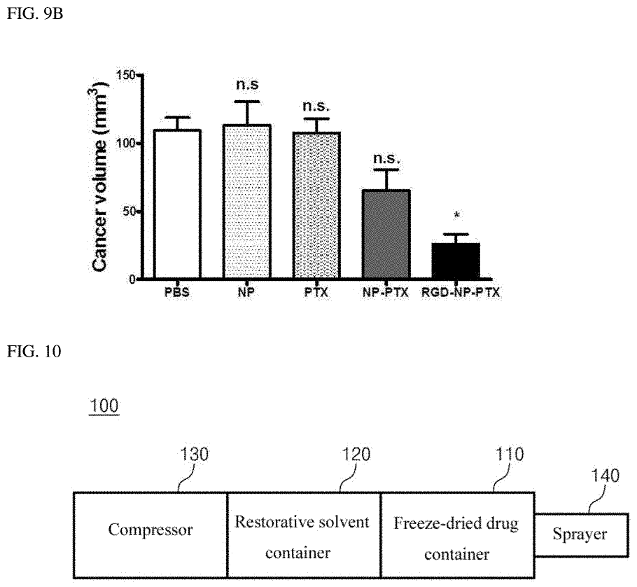

The drug delivery system for nose-to-brain delivery may be a known nebulizer. According to the present invention, the drug delivery system for nose-to-brain delivery may include, as shown in FIG. 10, a freeze-dried drug container 110 for storing a freeze-dried drug, a restorative solvent container 120 for storing a solvent for thawing the freeze-dried drug, a membrane for preventing the freeze-dried drug from being mixed with the solvent and a compressor 130 which provides propulsion. The propulsion of the compressor may allow the membrane to be open, thereby mixing the freeze-dried drug with the solvent to thaw the freeze-dried drug, and allow the thawed drug to be sprayed.

In addition, the drug delivery system for nose-to-brain delivery may include a sprayer 140 for spraying a drug, and the drug may be sprayed to the outside using the sprayer.

The drug delivery system for nose-to-brain delivery may be configured to sequentially have the restorative solvent container, the membrane, the freeze-dried drug container and the sprayer based on the compressor.

The restorative solvent container is flexible, and the membrane may be open due to an inner pressure increased by modifying the restorative solvent container due to the propulsion. That is, as the restorative solvent container is transferred in the freeze-dried drug container direction due to the propulsion, the inner pressure of the restorative solvent container may be increased to open the membrane.

The compressor may provide propulsion to one end of the restorative solvent container, and the other end of the restorative solvent container may be blocked from the freeze-dried drug container by the membrane.

Therefore, one end of the restorative solvent container may include an accommodation groove to which the propulsion is provided.

The freeze-dried drug container 110 may store a drug in a freeze-dried state. The freeze-drying of a drug may mean that, after the drug is frozen, an ambient pressure is lowered to evaporate water in a solid state into gas. That is, the freeze-dried drug container 110 may store a drug as a freeze-dried powder. In the present invention, the anticancer agent-containing NPs may be stored by freeze-drying.

The freeze-dried drug container may include micropores for spraying the thawed drug. Therefore, the drug sprayed from the micropores may be sprayed to the outside through the sprayer.

The membrane may prevent mixing of the freeze-dried drug and the solvent in drug storage and may be open in drug spraying, and thus may be configured to thaw the freeze-dried drug when mixed with the solvent.

The restorative solvent container 120 may store a restorative solvent which thaws the freeze-dried drug. Hereinafter, the restorative solvent may be abbreviated as a solvent for the sake of convenience.

The restorative solvent container 120 may contain at least one of, for example, glycerol, propylene glycol, polyethylene glycol, polypropylene glycol, ethyl alcohol, isopropyl alcohol, peanut oil, sterile water, a sterile normal saline solution and a sterile phosphate buffer solution as the restorative solvent.

In a drug-storing mode, the freeze-dried drug in the freeze-dried drug container 110 and the restorative solvent in the restorative solvent container 120 may be prevented from being mixed by the membrane. In contrast, in a drug-spraying mode, the membrane is open, such that the freeze-dried drug of the freeze-dried drug container 110 and the restorative solvent of the restorative solvent container 120 may be mixed, and the freeze-dried drug may be thawed and restored.

The compressor 130 may provide propulsion to the drug delivery system 100. More specifically, the compressor 130 may provide propulsion for spraying a drug, and opening or breaking the membrane between the freeze-dried drug container 110 and the restorative solvent container 120, thereby mixing the freeze-dried drug with the restorative solvent.

The compressor 130 may be operated in various ways. The compressor 130 may be formed as a syringe type such that an operator can directly provide propulsion. Unlike this, the compressor 130 contains compressed gas to be sprayed by manipulation of the operator, such that propulsion is provided. Hereinafter, for the sake of convenience, the compressor 130 is considered to contain compressed gas.

The compressed gas may consist of a material which is safe to be inhaled into a human body. For example, the compressed gas may consist of at least one of a hydrofluoroalkane (HFA), nitrogen, a chlorofluorocarbon (CFC), and air.

The compressed gas is not necessarily compressed gas, and may also be provided as a compressed liquid.

The sprayer 140 may provide a path for spraying a thawed drug prepared by mixing a freeze-dried drug with a restorative solvent through propulsion provided from the compressor 130.

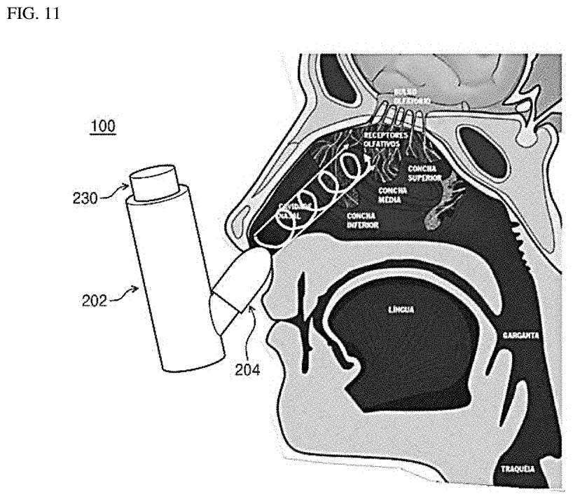

FIG. 11 is a diagram illustrating a method of using a drug delivery system for nose-to-brain administration of the present invention. The drug delivery system 100 for nose-to-brain delivery, shown in FIG. 11, may include a first housing 202 containing a compressor 230; and a second housing 204 containing a freeze-dried drug container, a restorative solvent container, a membrane and a sprayer.

Referring to FIG. 11, one end of the drug delivery system 100 of the present invention may be input into a nose. While the end of the drug delivery system 100 is input into the nose, the drug stored in a freeze-dried state may be thawed and sprayed into the nose in a helical form (see the white arrow) by pushing the compressor 230. As the drug sprayed into the nose reaches the right spot of the nose-to-brain drug delivery, a nose-to-brain drug delivery rate may be enhanced.

In addition, when the drug delivery system of the present invention is used while a subject, for example, the head of a subject is maintained in the Mecca position, a nose-to-brain drug delivery effect may be maximized. Drug injection in the Mecca position may provide an effect of preventing the input of the drug into another organ by intensively delivering the drug through the nose. Here, the Mecca position may refer to a position in which the head of a subject faces the chest thereof.

In addition, when the nose-to-brain drug delivery system of the present invention is used while a subject is sleeping, anesthetized or unconscious, the drug may be effectively provided by inducing the above-described Mecca position.

The present invention also provides a method of treating a brain disease, which includes intranasally administering the intranasal pharmaceutical composition for treating a brain disease to a subject in need thereof.

The intranasal pharmaceutical composition for treating a brain disease may be intranasally administered while the composition is contained in an injection device including a container which can contain the composition.

According to an exemplary embodiment of the present invention, the composition may be intranasally administered by being contained in a freeze-dried drug container of the above-described nose-to-brain drug delivery system.

The intranasal administration may be performed while a subject is sleeping, anesthetized or unconscious.

The brain disease may be a brain tumor.

The subject may be, but is not limited to, a mammal such as a dog, a cat, a rat, a mouse or a human.

While the present invention has been described in detail with reference to preferable examples, the scope of the present invention is not limited to specific examples, and should be interpreted by the accompanying claims. In addition, it should be understood by those of ordinary skill in the art that the present invention can be modified and altered in various ways without departing from the scope of the present invention.

### EXAMPLES

### <Example 1> Preparation of PTX-Loaded PLGA NPs

For PTX delivery, PLGA NPs were used. In addition, to improve effective drug release and target specificity in cancer environments, the surface of NPs was modified with an RGD peptide. The RGD peptide targets an integrin receptor expressed by malignant cancer cells.

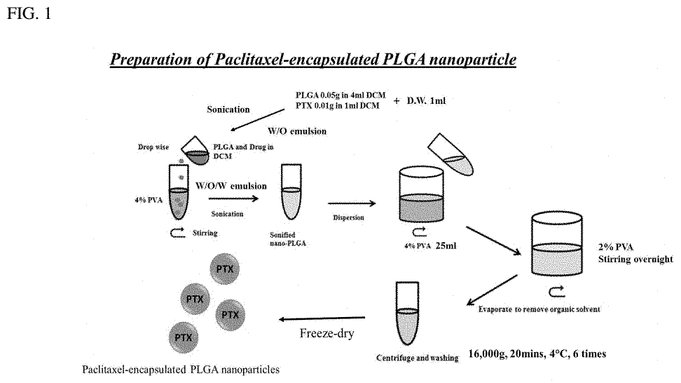

To this end, PTX-loaded PLGA NPs (NP-PTX) were prepared by a water-in-oil-in-water (w/o/w) double emulsion method (F, Danhier et al. Journal of Controlled release, vol. 133(1), pp. 11-17, 2009), which has been described above. PTX (1%, w/v) and PLGA (4%, w/v) were dissolved in dichloromethane, and deionized water was added to the resulting solution at a volume ratio of 1:5, followed by emulsification using a probe-type sonicator (Branson Digital Sonifier, Danbury, Conn.) with a power output of 25 W for 60 seconds at room temperature. The single emulsion (w/o) was re-emulsified in an aqueous PVA solution (4%, w/v), and sonicated at 30 W for 120 seconds (w/o/w). The double emulsion was poured in a PVA (1%, w/v) solution, and stirred overnight to evaporate the solvent. NP-PTX was obtained through centrifugation at 16000 rpm, washed, and freeze-dried (FIG. 1).

### <Example 2> Physicochemical Characterization of NPs

A Z-average size of the NP-PTX prepared as described above was measured with Malvern's Zetasizer Nano ZS (Malvern Instruments, Worcestershire, UK). 1 mg of NPs were dissolved in 1 mL of filtrated deionized water. Five detected values of the Z-average size (nm) and the polydispersity (25° C., each measured at 170°) were used. For data analysis, the Z-average size was determined using the viscosity (0.8872 mPa·s) and refractive index (1.33) of water. The morphology of the surface of NPs was examined with a scanning electron microscope (Tokyo, Japan). The NPs were suspended in deionized water (0.5% w/v), and mounted on an aluminum holder at room temperature. The mounted sample was dried overnight, and coated with platinum under a vacuum.

Subsequently, the drug loading efficiency and release profile of NP-PTX were measured by HPLC (Waters HPLC model). The column was a symmetric C18 column (100 Å, 5 μm, 4.6 mm×250 mm). A mobile phase was acetonitrile/water (75/25 v/v), a flow rate was maintained at 1 mL/min, and the chromatographic result was detected at a wavelength of 227 nm. To determine the loading efficiency, 50 μl of NPs were dissolved in a 1N NaOH solution and the NaOH solution was neutralized with a 1N HCl solution. Acetonitrile was added to a PTX solution in which PTX was dissolved, thereby dissolving PTX. After the loading efficiency of the PTX-loaded PLGA NPs was measured, 0.5 mg of the PTX-loaded PLGA NPs were dispersed in 5 mL of a phosphate buffered solution (PBS, pH 7.4) and incubated at 37° C. while stirred at the determination time, and the resulting solution was ultra-centrifuged at 22,000 g and 4° C. for 30 minutes. The supernatant was collected and mixed with 5 mL of acetonitrile, the pellet was resuspended with 5 mL of PBS and incubated again while stirring at 37° C. Each sample was injected at a volume of 50 μL, and analyzed under the above-described HPLC conditions.

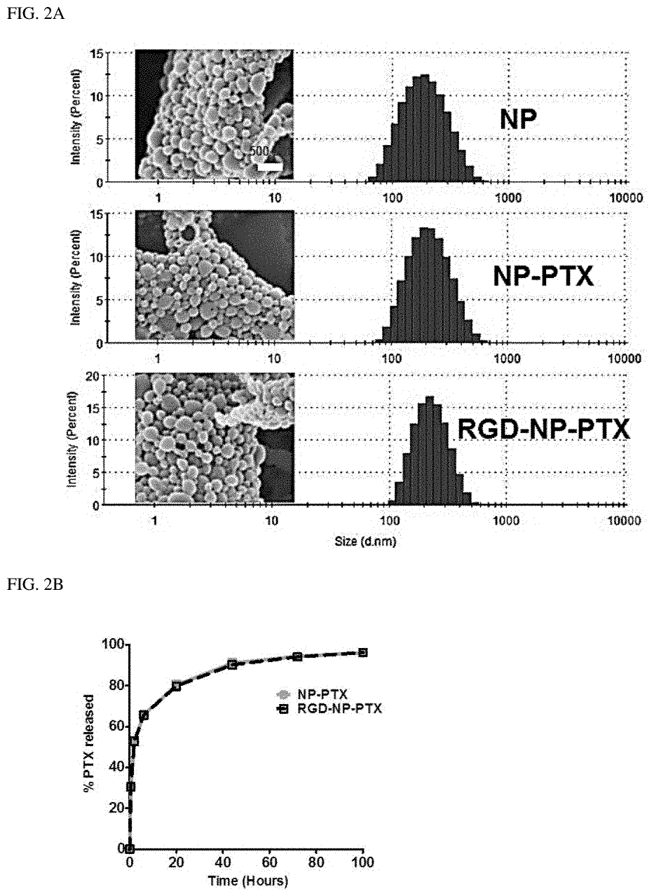

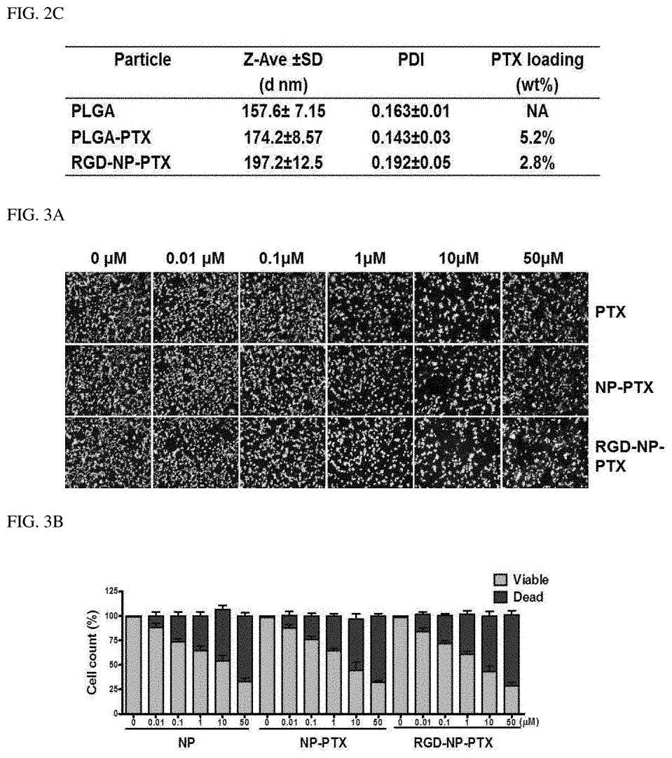

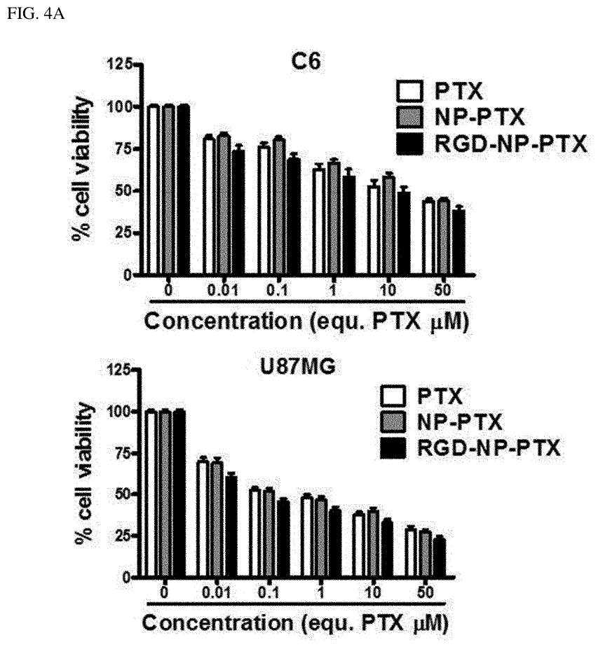

A size of the NPs prepared in Example 1 was approximately 150 to 200 nm in an acceptable narrow distribution. The polydispersity index was PDI≥0.1, and did not have much difference between before and after PTX loading or before and after surface modification using an RGD peptide (FIGS. 2A and 2C). This result was consistent with that of the conventional research in which PTX loading in NPs does not affect their size, compared with drug-free NPs. The same pattern was also observed by scanning electron microscopy. An image obtained by scanning electron microscopy showed the formation of uniform spherical particles (FIG. 2A).

In previous research, NPs having a size of less than 230 nm indicated improved cell delivery both in vitro and in vivo.

A content ratio of PTX loaded in NPs was less than approximately 5.3%, and encapsulation efficiency was approximately 40%. However, in the RGD-NP-PTX group, the drug content ratio was ultimately decreased to less than 2.8%, and the encapsulation efficiency was also decreased to 30%, showing that a loosely-encapsulated drug was released (FIG. 2C).

Within the initial 30 minutes, approximately 30% of the drug was released from the NPs, and then gradually released for more than 4 days (FIG. 2B). The continuous release of PTX will be advantageous in terms of an anti-tumor effect at a site requiring a considerable content of the drug in an intracellular environment. Consequently, this result demonstrates that hydrophobic PTX drug loading into the NPs formed uniform spherical NPs, which makes it possible to continuously release encapsulated PTX.

### <Example 3> Anti-Tumor Effect of PTX-Loaded NPs by Inhibition of Cell Proliferation

PTX is one of the widely used anti-tumor drugs used for some types of solid cancer. In the cultured C-6 glioma cells, to identify the anti-cancer effect of PTX, PTX only, NP-PTX and RGD-NP-PTX were treated with various micromolar (μM) concentrations.

The rat (C6) and human (U87MG) glioblastoma cells used in this experiment were obtained from ATCC (Rockville, Md.), and cultured in Dulbecco's Modified Eagle's Medium (DMEM) containing 10% fetal bovine serum, penicillin (100 IU/mL) and streptomycin (100 μg/mL) at 37° C. in a 5% CO₂ incubator. For all in vitro experiments, the cells were seeded in a 12-well plate at a density of 1×10⁵ cells/well. Subsequently, the cells were treated with various concentrations of PTX only or an equivalent concentration of NP-PTX for 24 hours.

For cell cycle analysis, the C6 or U87MG glioblastoma cells were exposed to various concentrations of PTX only or an equivalent concentration of NP-PTX for 24 hours. And then, the cells were collected, and fixed with 70% ethanol at 4° C. for 2 hours. After incubation, the cells were washed, and additionally incubated with a DNase-free RNase (1 mg/mL) and 0.02 mg/mL of propidium iodide (PI). A cell cycle profile was studied using a flow cytometer (BD FACS Calibur™), and analyzed using FlowJo software.

To analyze the viability and apoptosis of the C6 glioma cells, the cells were cultured as described above, treated with PTX or NP-PTX for 24 hours, and cultured with calcine-AM and an ethidium homo dimer (EthD-1) using a viability/apoptosis potential/cytotoxicity kit (Thermo Fisher Scientific, Waltham, Mass.) according to the manufacturer's instructions. An image was captured using a fluorescence microscope (Leica, Wetzlar, Germany). A percentage of live or dead cells was calculated using ImageJ software, developed by the National Institute of Health (NIH).

To test an in vitro anti-tumor effect, the anti-tumor efficacy of PTX or NP-PTX was measured in a 24-hour incubator by CCK-8 analysis (Dojindo Laboratories, Kumamoto, Japan) at an indicated concentration for 24 hours according to the manufacturer's instructions. To confirm the percentage of dead cells after treatment with PTX or NP-PTX, the cells were stained with a PE annexin V apoptosis detection kit (BD Pharmingen™) according to the manufacturer's instructions.

To produce a stable C6 cell line (C6-Luc) which expresses luciferase, a luciferase-expressing lentivirus vector (RediFect Red-FLuc-GFP, PerkinElmer, Waltham, Mass.) was used. The cell line was treated with Red-FLuc-GFP at 37° C. for 8 hours. A plate was further cultured at 37° C. for 48 hours. Until the stable luciferase-expressing cell line was established, the cells were classified by GFP expression using an FACS analyzer.

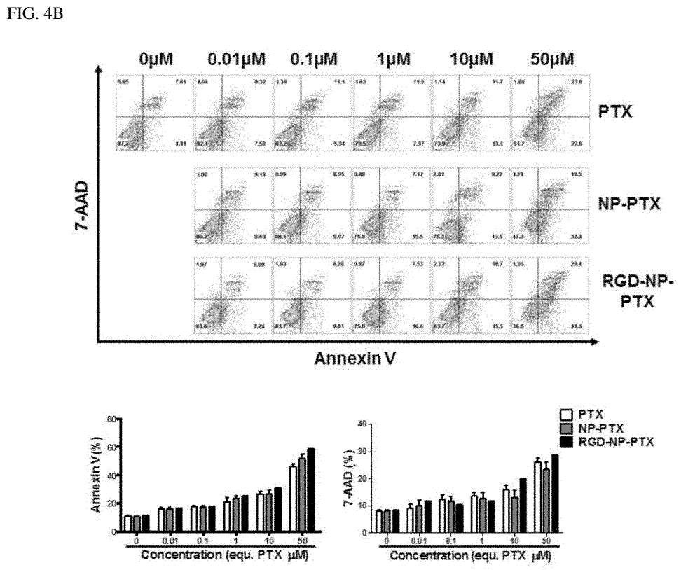

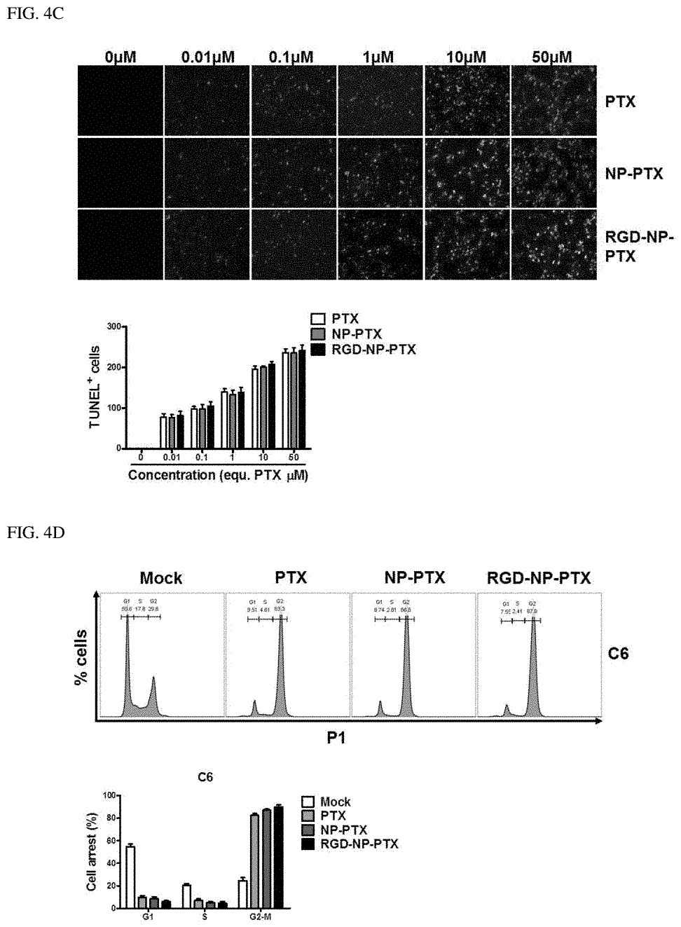

As shown in viability and apoptosis analyses, cancer cell death is increased in a concentration-dependent manner (FIG. 3A). In addition, in treatment with PTX only, 8.3%, 23.1%, 31.8%, 49.2% and 63.8% of dead cells were found at 0.01, 0.1, 1, 10 and 50 μM, respectively. The cancer cell death behavior of NP-PTX was relatively similar to the treatment with PTX only after 24 hours. However, in treatment with RGD-NP-PT, the cancer cell death behavior was slightly increased (FIG. 3B).

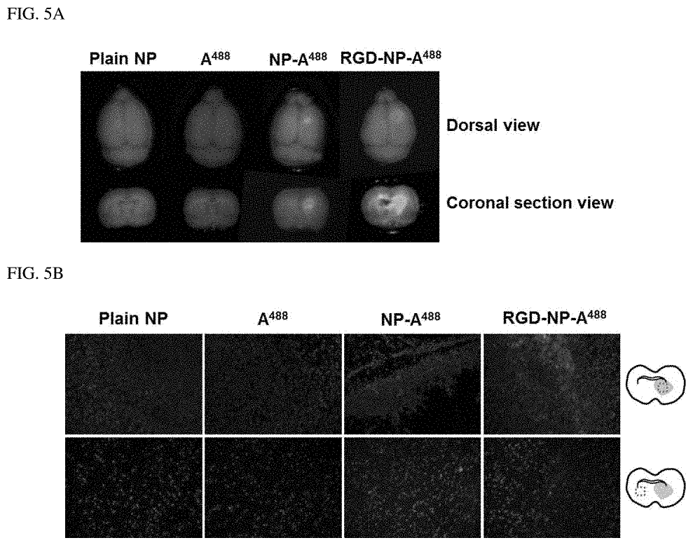

Similar to the viability and apoptosis analysis, CCK-8 analysis also showed a similar pattern of the anti-proliferation effect at each treatment concentration in C6 or U87MG cells (FIG. 4A). C6-glioma cell proliferation was dramatically decreased to less than 35% in C6 cells and less than 20% in U87MG glioblastoma cells when an equivalent amount (50 μM) of PTX was treated in each case, compared to non-treated highly proliferating cells.

Subsequently, PTX-induced apoptosis in rat glioma cells was investigated by staining the cells with annexin V and 7-AAD as markers for early and late apoptosis, respectively. The number of both annexin V and 7-AAD-positive cells were concentration-dependently increased after treatment with PTX, NP-PTX or RGD-NP-PTX (FIG. 4B). In addition, when PTX was treated at 0.01, 0.1, 1, 10 and 50 μM, the annexin V-positive cells were increased to 22.5%, 24.1%, 30.1%, 33.1% and 34.2%, respectively (FIG. 4B, lower left panel). As a drug concentration was elevated in cells treated with an equivalent amount of PTX, the number of 7-AAD-positive cells was also increased (FIG. 4B, lower right panel).

Additionally, a TUNEL assay was performed to examine the total number of apoptotic cells in PTX-, NP-PTX- or RGD-NP-PTX-treated C6-glioma cells. The number of apoptotic cells stained with TUNEL was concentration-dependently increased in all groups treated with an equivalent amount of PTX (FIG. 4C). The number of TUNEL-positive cells was estimated to be approximately 77, 97.5, 136.6, 198.6 and 236.1 at each tested micromolar concentration of PTX.

Hoechst staining showed a homogenous nuclei structure without segmentation or fragmentation in normal non-treated glioma cells. In contrast, the treatment with PTX, NP-PTX or RGD-NP-PTX resulted in severe formation of DNA fragmentation or segmentation or condensed nuclei (FIG. 4C).

These results suggest that PTX-loaded NPs have an effective anti-cancer effect against cultured glioma cells. In fact, consistent with the mechanism of action of PTX, the effective anti-cancer effect may be observed for a longer incubation period where most of the cells enter the G2 and M phases of the cell cycle.

Then, the effect of PTX treatment on the arrest of the cell cycle of rat and human glioma cells was confirmed. It has been well known that PTX exposure to cultured cells induces G2-M cycle arrest. The analysis of the DNA content of cells treated with PTX reveals the accumulation of a cell population arrested at the G2-M phase with a significant decrease of cells arrested at the G1 phase (FIG. 4D). According to the representative histogram data, 29% of the limited populations in non-treated cells are at the G2-M phase, and the majority of the cell population was in the G1 phase. When PTX only-, NP-PTX- or RGD-NP-PTX-treated C6 glioblastoma cells were treated with an equivalent amount of PTX (10 μM), 83%, 86% or 88% progressed to the G2-M phase and 9%, 8% or 7% at the G1 phase were exhibited (FIG. 4D). A similar pattern was also observed in human U87MG glioma cells having a slightly higher arrested population at the G2-M phase, suggesting a greater PTX response in the U87MG cells (FIG. 4D). Interestingly, compared with treatment with PTX only, the arrested G2-M checkpoint population of the glioma cells was slightly increased such that intracellular penetration of the PTX concentration was enhanced. This result demonstrated that PTX exposure to the cultured glioblastoma ultimately promotes cell cycle arrest at the G2-M phase.

### <Example 4> Delivery of NPs to Brain by Intranasal Inoculation

To evaluate the in vivo distribution of NPs in a glioma-bearing brain, alexa488 (A488) was conjugated to NH₂-modified PLGA NPs. Further, to achieve cancer-specific targeting of NPs, the A488-conjugated NPs were additionally modified with RGD. To confirm the in vivo distribution of the A488-conjugated NPs, a total of 100 μg of the A₄₈₈-conjugated NPs was intranasally inoculated in each nostril at the final volume of 25 μl using a POD device (refer to FIG. 11). At 24 hours after inoculation, the animal was sacrificed, and then an organ was dissected. The organ was washed with cold PBS, and the surface meninx was removed to eliminate self-fluorescence. The brain was observed to detect a fluorescent signal under an image station (Carestream, Rochester, N.Y.). The relative fluorescent intensity was measured using ImageJ software (NIH). To measure a cell density (%) in a glioma region, a single cell suspension was prepared using a 70-μm cell strainer (BD, Franklin Lakes, N.J.). The cells were collected using a flow cytometer (BD, Franklin Lakes, N.J.), and analyzed using FlowJo software.

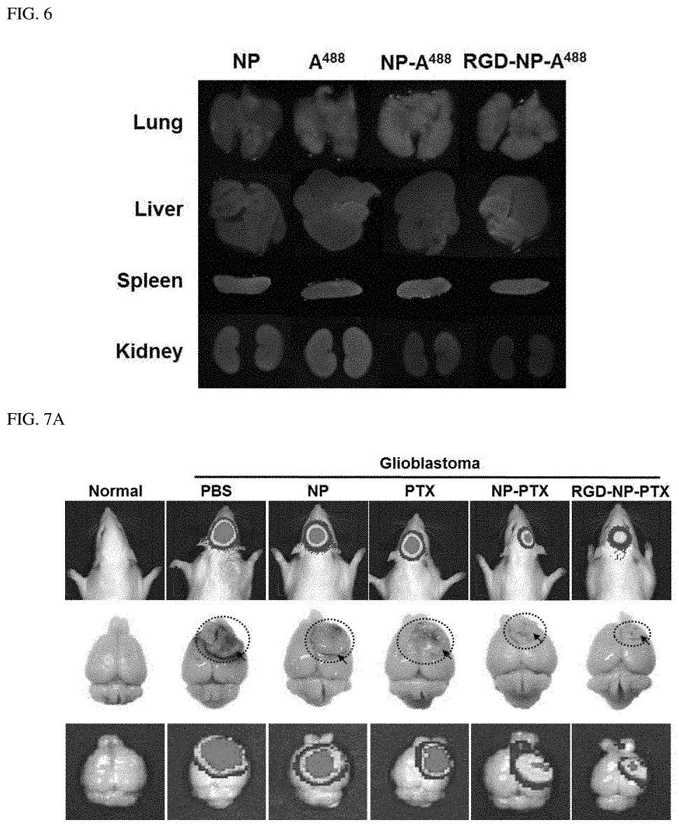

A single intranasal inoculation of RGD-modified NPs resulted in noticeable localization of fluorescent signals, specifically in the glioma region of the brain, at 24 hours after inoculation (FIG. 5A). The coronal brain section image showed an intensive distribution of A₄₈₈-labeled RGD-NPs, specifically in the cancer region, suggesting that the particles were localized in an integrin-rich cancer region. In contrast, the NP-A₄₈₈ group showed a poor distribution without localization in a cancer-specific region, suggesting limited tumor cell penetration.

Further, in the glioma-bearing animal brain, to evaluate a NP distribution, frozen sections were prepared. The fluorescent microscopy data revealed distinctive distribution patterns of NP-A₄₈₈ and RGD-NP-A₄₈₈ in the cancer region (FIG. 5B). Consistent with ex vivo brain imaging data, A₄₈₈ or NP-A₄₈₈ showed a poor distribution in the cancer region. In contrast, RGD-NP-A₄₈₈ was strongly localized in the cancer region, compared with a non-cancer region, suggesting that modification of NPs with an RGD peptide improves tumor cell internalization, specifically in the glioma region, rather than a non-glioma region (FIG. 5B).

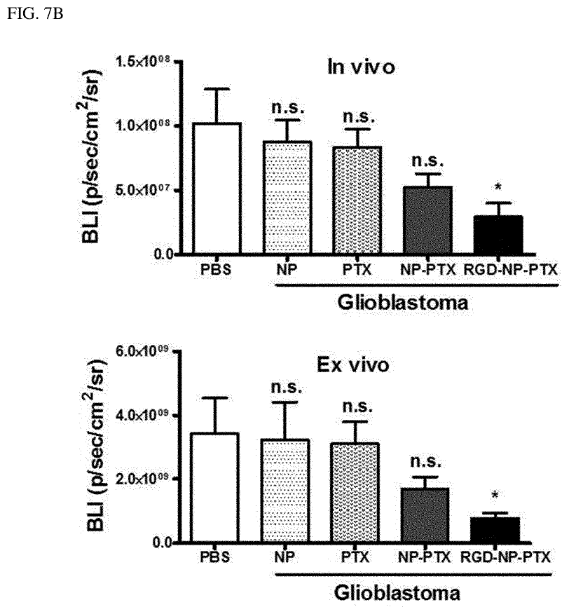

Although weak localization in the RGD-NP-A₄₈₈ treated group suggests continuous release of non-targeting NPs from the brain to the surroundings, the peripheral organ data showed localization in the liver and the kidney in the NP-A₄₈₈ treated group (FIG. 6).

### <Example 5> Effect of Decreasing Tumor Growth by Intranasally Inoculated PTX-Loaded NPs in Rat Glioblastoma Model

The therapeutic efficacy of intranasally-delivered PTX-loaded NPs was evaluated in an intracranial C6-Luc orthotropic model. The intranasal inoculation of the chromophore-dissolved PTX (Taxol) induced abnormal animal behavior within few minutes after inoculation probably due to its stickiness. Therefore, as a PTX solvent, DMSO was used. In addition, to minimize DMSO-associated cytotoxicity, the PTX-loaded NPs were dissolved in PBS. The intranasal inoculation started at day 4 after tumor administration and was performed daily for a total of three times.

Specifically, intracranial tumor models were established from 6-week-old male Sprague-Dawley rats. The anesthetized rats were placed in a stereotaxic frame, and the skull was gently exposed to spot the bregma. The monitoring points for the bregma were as follows: anteroposterior, 0 mm; lateral, 2.0 mm; and ventral, 4.0 mm. A fine burr hole (0.7 mm) was created in the skull without disrupting the dura using a microsurgical drill. A total of 2×10⁵/10 μl C6-Fluc cells were injected using a 26-gauge Hamilton microsyringe (80330; Reno, Nev., USA) at a speed of 0.9 μl/min. To generate a human glioblastoma model, 1×105/4 mL of U87MG-Fluc cells were inoculated into immunodeficient nude mice by the same method. After the operation, the skin was sutured. And then, the animals were randomly assigned to each group, 2 mg/kg of PTX was inoculated into the rats, or 1 mg/kg of PTX was intranasally inoculated into mouse models.

In vivo and ex vivo bioluminescence imaging was performed. For in vivo bioluminescence imaging, 150 mg/kg of D-luciferin (Caliper, Hopkinton, Mass.) was intraperitoneally injected into anesthetized animals. The live images were taken 15 minutes after the injection of D-luciferin by the IVIS Lumina caliper series (Life Technologies, Carlsbad, Calif.). To evaluate ex vivo bioluminescence, the excised brain tissue was incubated with a D-luciferin substrate for 15 minutes, followed by imaging as described above.

For Nissl staining, paraffin-embedded brain sections were deparaffinized, rehydrated, and then subjected to treatment with a 0.1% crystal violet solution according to a standard protocol. The stained sections were covered with cover slips, and randomly photographed using an optical microscope. The cancer volume was calculated using ImageJ software as described above.

For histological and TUNEL assays, paraffin-embedded brain sections were de-paraffinized, rehydrated, and then subjected to H & E staining. Afterward, H & E-stained sections were covered with cover slips, and observed under an optical microscope.

To investigate apoptosis in the de-paraffinized and hydrated brain sections, the TUNEL assay was performed using an in situ apoptosis detection kit (Millipore) according to the manufacturer's instruction. The nuclei were counterstained with Hoechst 33342, and mounted with an aqueous mounting solution (Abcam, Cambridge, UK). Fluorescent signals of the cells were measured by taking images using a fluorescence microscope (Leica, Wetzlar, Germany).

For immunohistochemical staining, sections were inactivated with a pre-warmed antigen retrieval buffer (10 mM sodium citrate, 0.05% Tween-20 (w/v), pH 6.0) through thermal treatment for 25 minutes at 95° C., and cooled at room temperature. Next, sections were blocked with TBST containing 1% BSA and 10% goat serum for 1 hour at 37° C., and incubated with Ki67 primary antibody (Abcam, Cambridge, UK) overnight at 4° C. Afterward, sections were washed with TBST, and a secondary polyclonal antibody coupled to HRP was applied for 2 hours. The resulting product was washed with TBST 5 times, and then the sections were developed using a DAB substrate (GE Healthcare, Little Chalfont, UK).

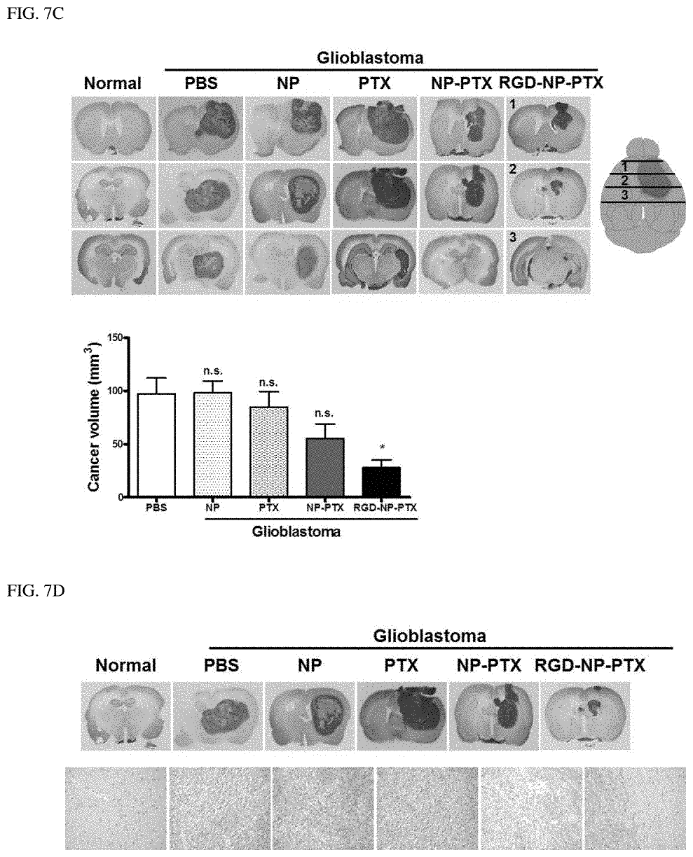

The tumor progression in saline- or PTX-free NP-treated groups was rapidly performed, and became severe at day 14 after the injection of glioma cells. The PTX only-treated group was relatively identical to the saline-treated group, and thus this suggests that the hydrophobic characteristic of PTX restricts sufficient drug penetration and accumulation in the tumor region. The treatment with NP-PTX or RGD-NP-PTX, however, interrupts cancer progression (FIG. 7A, upper panel).

At day 14, an average in vivo bioluminescence measurement showed a 1.0×10⁸-fold increase in bioluminescence signals in saline-, PTX only- or NP only-treated animals. In the NP-PTX-treated animal, tumor growth was marginally lowered to 47%, and this progression was significantly reduced to 70% in the RGD-NP-PTX treated group (FIG. 7B, left panel). Consistent with the in vivo bioluminescence data, the excised brain images showed a massive tumor lump in the PBS-, PTX-free NP- or PTX-only group 14 days after tumor implantation in contrast to either NP-PTX- or RGD-NP-PTX-treated group (FIG. 7A, middle and lower panels). Further, the bioluminescence signals of ex vivo brain samples showed 52% and 74% decreases in animals treated with NP-PTX or RGD-NP-PTX, respectively, compared with the PBS-treated animal (FIG. 7B, right panel).

A tumor volume in an animal intranasally inoculated with NP-PTX or RGD-NP-PTX was significantly smaller than that in the saline-treated group (FIG. 7C). Representative Nissl-stained brain coronal slices (three slices of each brain) showed no reduction in tumor size in PBS-, NP- and PTX-only inoculated animals, and tumor cells were equally distributed in all three brain coronal slices. The NP-PTX-inoculated groups show relatively completely suppressed tumor growth in an anterior brain slice (2.70 mm to bregma) and completely suppressed tumor growth in a posterior coronal section (−6.04 mm from bregma) (FIG. 7C). The tumor volumes (mm³) reached 98.4, 99.1 and 90.1 in the single PBS, NP, PTX-only inoculated animals, respectively. NP-PTX or RGD-BP-PTX treatment resulted in a reduction in this load to 52.2 and 27.6, respectively (FIG. 7C, lower panel). Compared with the saline-treated group, the tumor volumes were reduced by 44% and 72% in the NP-PTX and RGD-NP-PTX-inoculated groups, respectively. Generally, a consistent reduction in tumor load was observed and analyzed in vivo or ex vivo.

Representative H & E images showed a well-differentiated cell morphology from normal brain tissues, but cells in a tumor core became round to oval with an eosinophilic and compact arrangement of nuclei in all groups. However, the NP-PTX or RGD-NP-PTX-inoculated animals showed a large area of cell death and inhibited glioma cell growth, compared with saline-inoculated animals (FIG. 7D).

Further, when the TUNEL staining indicates a large number of apoptotic cells present in RGD-NP-PTX-inoculated animals, it suggests enhanced anti-tumor activity by inducing tumor cell death (FIG. 7E, upper).

The immunohistochemistry for cell proliferation assay shows a large number of Ki67⁺ cells in the tumor core of the saline-inoculated animals, but these cells considerably decrease in the NP-PTX or RGD-NP-PTX-inoculated group. Further, the inhibition of tumor cell influx populations is illustrated (FIG. 7E, lower).

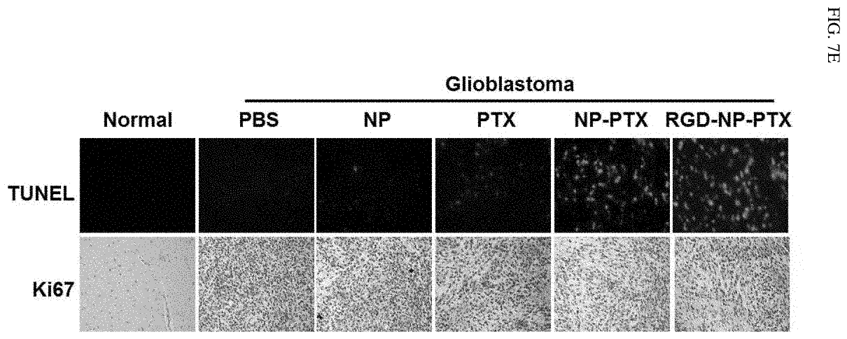

Further, the in vivo toxicity of NPs was evaluated by measuring a body weight of the inoculated animal. The change in body weight is reliable indicator for assessing the in vivo toxicity of a delivered drug. The RGD-NP-PTX-inoculated animals did not show any significant change in body weight, which was similar to saline- or NP-inoculated animals. However, an insignificant change in body weight was shown in all animals due to cancer mass over time after tumor implantation (FIG. 8). The PTX only-inoculated animals showed a significant effect on body weight loss, suggesting a toxic effect of PTX. As a result, the in vivo treatment data suggests that intranasal inoculation of NP-PTX reduces a tumor mass by inducing apoptosis.

### <Example 6> Decrease in Tumor Growth by Intranasally Inoculated PTX-Loaded NPs in Human Glioblastoma Model

To evaluate the clinically relevant therapeutic efficacy of PTX-loaded NPs, a highly invasive human glioblastoma pre-clinical model was selected.

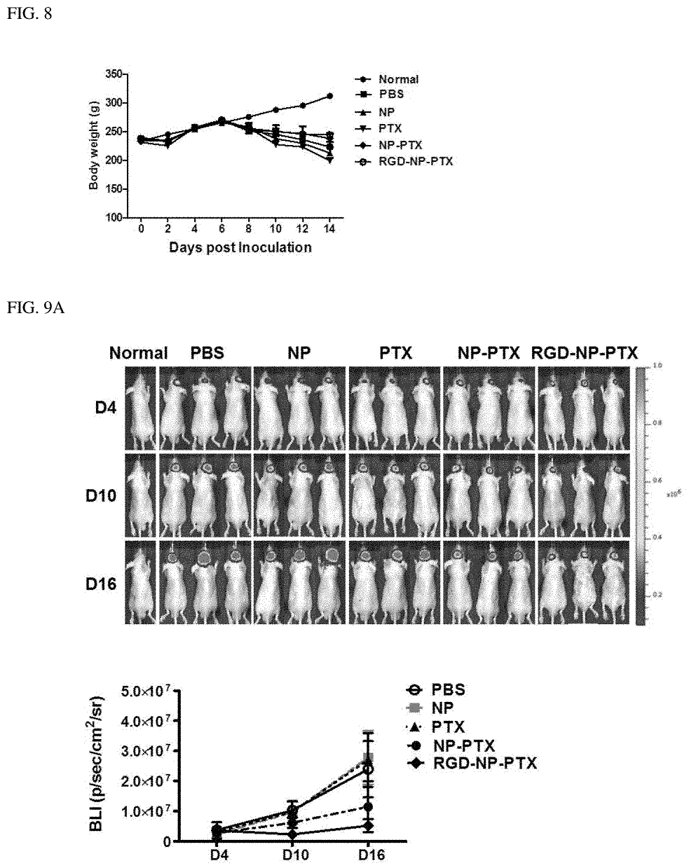

As a result of analysis of representative bioluminescence intensity (BLI) data, a tumor was equally initiated within 4 days after cell implantation in all tested groups. After a total of three intranasal inoculations, the tumor growth was effectively inhibited in RGD-NP-PTX despite a slight change in NP-PTX, when compared with the saline-treated group (FIG. 9A, upper). Consistent with the rat glioblastoma data, PTX-only treatment did not show any therapeutic effect, which indicates limited brain delivery. Compared with saline treatment, NP-PTX or RGD-NP-PTX treatment induces 41% or 77% reductions two days after the final treatment and 60% or 80% reductions 8 days after the final treatment in bioluminescence intensity, respectively (FIG. 9A, lower).

The decrease in bioluminescence intensity in NP-PTX at day 2 after the final inoculation can be caused by a PTX initial effect, but when the inoculation stops, cancer growth restarts. In contrast, the mice inoculated with RGD-NP-PTX continuously delay cancer progression over time. Further, as a result of the analysis of a tumor volume (mm³), the tumor volume was 75.6 within 16 days after tumor implantation, but NP-PTX and RGD-NP-PTX treatment resulted in 54% and 75% tumor reductions, respectively (FIG. 9B). The difference in therapeutic index of PTX-loaded NPs in a mouse to rat model may be caused by a gender or inoculation difference. In addition, the mouse has a relatively small surface area of the nose, and a substantially large amount of the olfactory epithelium in the nasal cavity allows better brain uptake. This result demonstrated that RGD-NP-PTX exhibited efficient inhibition of tumor growth, compared with other tested groups.

[Descriptions of reference numerals]

| | |
|---|---|
| 100: drug delivery system | 110: freeze-dried drug container |
| 120: restorative solvent container | 130, 230: compressor |
| 140: sprayer | 202: first housing |
| 204: second housing | |

The present invention may be applied in treatment of a brain tumor.

What is claimed is:

1. An intranasal pharmaceutical composition for treating a brain disease, comprising:
nanoparticles containing an anticancer agent for inhibiting the formation or breakdown of a mitotic spindle.

2. The composition according to claim 1, wherein the anticancer agent for inhibiting the formation or breakdown of a mitotic spindle includes one or more selected from the group consisting of vinblastine, vincristine, vinflunine, vindesine, vinorelbine, cabazitaxel, docetaxel, larotaxel, ortataxel, paclitaxel, tesetaxel and ixabepilone.

3. The composition according to claim 1, wherein the nanoparticles are formed from any one or more polymers selected form the group consisting of poly-D-lactic acid, poly-L-lactic acid, poly-D,L-lactic acid, poly-D-lactic acid-co-glycolic acid, poly-L-lactic acid-co-glycolic acid, poly-D,L-lactic acid-co-glycolic acid (PLGA), polylactide (PLA), polylactide-glycolide (PLA/GA), polyalkylcyanoacrylate, poly(acryloyl hydroxyethyl) starch, a copolymer of polybutylene terephthalate-polyethylene glycol, chitosan and a derivative thereof, a copolymer of polyorthoester-polyethylene glycol, a copolymer of polyethyleneglycol terephthalate-polybutylene terephthalate, polysebacic anhydride, pullulan and a derivative thereof, starch and a derivative thereof, cellulose acetate and a derivative thereof, polyanhydride, polycaprolactone, polycarbonate, polybutadiene, polyester, polyhydroxybutyric acid, polymethyl methacrylate, polymethacrylic acid ester, polyorthoester, polyvinylacetate, polyvinyl alcohol, polyvinyl butyral, polyvinyl formal, albumin, casein, collagen, fibrin, fibrinogen, gelatin, hemoglobin, transferrin, zein and a mixture thereof.

4. The composition according to claim 1, wherein a tumor marker-targeting component is additionally conjugated to the nanoparticles.

5. The composition according to claim 4, wherein the tumor marker-targeting component is any one or more selected from the group consisting of an RGD peptide, cilengitide, and an EGF- or EGFR-binding peptide.

6. A kit for intranasal administration for treating a brain disease, comprising:
the intranasal pharmaceutical composition for treating a brain disease of claim 1; and
a nose-to-brain drug delivery system.

7. The kit according to claim 6, wherein the intranasal administration is carried out on a subject which is sleeping, anesthetized, or unconscious.

8. A method of treating a brain disease, comprising:
intranasally administering the intranasal pharmaceutical composition for treating a brain disease of claim 1 to a subject in need thereof.

9. The method according to claim 8, wherein the intranasal administration is carried out on a subject which is sleeping, anesthetized, or unconscious.

10. The method according to claim 8, wherein the brain disease includes a brain tumor.
